(19) United States
(12) Patent Application Publication
(10) Pub. No.: US 2008/0206151 A1
(43) Pub. Date: Aug. 28, 2008

(54) Liposomes

(76) Inventor: Alan Cuthbertson, Oslo (NO)

Correspondence Address: GE HEALTHCARE, INC., IP DEPARTMENT, 101 CARNEGIE CENTER, PRINCETON, NJ, 08540-6231, US

(21) Appl. No.: 11/912,206

(22) PCT Filed: Apr. 25, 2006
(86) PCT No.: PCT/NO2006/000157

§ 371 Date: Oct. 22, 2007

(30) Foreign Application Priority Data
Apr. 25, 2005 (NO) 20051995

(51) Int. Cl. A61K 49/18; A61K 9/127; C07F 9/06

(52) U.S. Cl. 424/9.36; 424/450; 564/15; 424/9.3

(57) ABSTRACT

The present invention relates to a process for the manufacture of targeting liposomes comprising vector compounds conjugated to the hydrophilic part of modified phospholipids. The present invention provides the modified phospholipids and liposomes containing said modified phospholipids.

### FIELD OF THE INVENTION

The present invention relates to a novel process for the manufacture of targeting liposomes comprising vector compounds conjugated to the hydrophilic part of the liposome. The invention includes a modified phospholipid for use as membrane material in the manufacturing of the liposomes and also a modified phospholipid binding a targeting vector. Liposomes of the invention also can carry a paramagnetic metal at the surface making the liposomes useful as diagnostic contrast agent for use in Magnetic Resonance Imaging, MRI.

### BACKGROUND TO THE INVENTION

Liposomes are vesicles consisting of a phospholipid bilayer or multilayer enclosing an aqueous interior. Encapsulation of material in the aqueous interior enables the accumulation of that material in target tissues and decreases its spread to non-target tissues where it might be harmful. This is an especially useful mechanism where the material is a drug with toxic side effects.

Liposomes are also of considerable interest because of their value as carriers for diagnostic agents. Examples are diagnostic agents for magnetic resonance imaging (MRI), single photon emission tomography (SPECT), ultrasound and x-ray.

Liposomal contrast agents for use in ultrasound imaging are described in e.g. WO90/04943 and WO91/09629, both of which disclose gas encapsulating liposomes and WO91/09629 which discloses a range of materials from which the gas lipid membrane in such liposomes may be formed.

WO88/09165 describes liposome preparations for injection containing an X-ray contrast agent solution within the liposomes and a buffered physiologically saline continuous phase in which the liposomes are suspended.

WO 02/089771 discloses liposomes containing internalized material for imaging purposes.

WO 98/18500 and WO 98/18501 are both concerned with targetable diagnostic and/or therapeutically active agents, e.g. ultrasound contrast agents where targeting vectors are linked to the surface of gas-filled microbubbles.

Contrast agents targeting specific receptors or tissues, particularly receptors associated with disease or diseased tissues, are gaining importance in diagnostic imaging. Biologically active molecules which selectively interact with specific receptors or cell types are useful for the retention of imageable moieties or reporters to target. Peptides are of particular important biologically active molecules useful as targeting moieties. Using peptides as targeting moieties in contrast agents entail that considerable consideration have to be taken in manufacturing procedures to prevent conditions that may cause denaturation of peptides. Denaturated peptides may loose their targeting specificity and ability to bind to specific cell types or receptors.

Liposomes are prepared under hash conditions such as e.g. high temperature (60° C. and above) that can lead to the denaturation of peptides. This problem can be solved by preparing the liposomes before the targeting peptide is attached to the surface, however there are difficulties related to this approach such as appropriate and available binding sites on the liposome surface for the attachment. The present invention solves this problem by comprising amine containing phospholipids with functional groups in the liposome membrane. Functional groups that are useful as sites for binding of e.g. peptides are exposed at the liposome surface.

### THE PRESENT INVENTION

In the manufacturing of liposomes conjugated with vectors there exist a problem that vectors, particularly of peptidic nature, are vulnerable under the conditions of which liposomes are formed. Vectors of this nature that are exposed to the hash conditions under which liposomes are formed may break up, denaturalise or change in other ways such that they loose their characteristic features as vectors e.g. receptor binding affinity and specificity.

This problem is solved by the present invention where liposomes are prepared with modified phospholipids in the membrane and then conjugating vectors to the modified phospholipids in the liposomes under conditions tolerable for the vectors.

The present invention provides a process for the manufacturing of targeting liposomes where liposomes having amine containing phospholipids comprising functional groups comprised in the liposome membrane are conjugated to targeting moieties, e.g. peptides or antibodies, containing a counter functional group to the functional groups exposed in the liposomes.

The invention also provides a modified phospholipid for use in the manufacturing of targeting liposomes where said phospholipid contains a functional group at its hydrophilic part.

The present invention further provides targeting moieties containing a counter functional group to the functional groups exposed at the liposome surface.

Pharmaceutical compositions comprising the liposome of the invention, use and methods of imaging also form part of the invention.

### DETAILED DESCRIPTION OF THE INVENTION

In a first aspect, the present invention provides a process for the manufacture of targeting liposomes comprising vector compounds conjugated to the hydrophilic part of modified phospholipids comprising the steps of

(a) reacting an amine containing phospholipid with a group R¹-X wherein R¹ is a functional group R^{1a} or R^{1b} where

R^{1a} is selected from an aldehyde moiety, a ketone moiety, a protected aldehyde as an acetal, a protected ketone such as a ketal, or a functionality such as diol or N-terminal serine residue, which can be oxidised to an aldehyde or ketone using an oxidising agent and

R^{1b} is selected from primary amine, secondary amine, hydroxylamine, hydrazine, hydrazide, aminoxy, phenylhydrazine, semicarbazide or thiosemicarbazide group and X is a reactive group that in the reaction with the amine of the phospholipid forms an amide bond by which a modified phospholipid containing a functional group R¹ is formed, (b) forming liposomes optionally comprising in vivo imageable moieties bound to the membrane from a mixture comprising the modified phospholipids from (a) in a conventional manner, and

(c) reacting the R¹ functional groups of the modified phospholipids of the liposomes with a group R²-Y wherein R² is a functional group R^{2a} or R^{2b} where

R^{2a} is selected from primary amine, secondary amine, hydroxylamine, hydrazine, hydrazide, aminoxy, phenylhydrazine, semicarbazide or thiosemicarbazide group and

R^{2b} is selected from an aldehyde moiety, a ketone moiety, a protected aldehyde as an acetal, a protected ketone such as a ketal, or a functionality such as diol or N-terminal serine residue, which can be oxidised to an aldehyde or ketone using an oxidising agent, and

Y is a vector,

to form targeting liposomes.

In a first step (a) an amine containing phospholipid is reacted with a group R¹-X to form a modified phospholipid where R¹ is bound to the hydrophilic part of the phospholipid. X is a group comprising an acid, an anhydride or an ester functionality. The amine (—NH₂) of the phospholipid is reacted with X (—COOH, —(CO)O(CO)—, —C—O—O—C—) to form a amide bond.

In a second step (b) of the process the modified phospholipids from step (a) are mixed with other suitable phospholipids and liposomes of the invention can be prepared by any conventional procedures used for formation of liposomes. These preparation procedures include the Bangham method (J. Mol. Dial. 13, 238-252, 1965), the polyvalent alcohol method (Japanese Examined Patent Publication (Kokoku) No. 4-36734), the lipid-solution method (Japanese Examined Patent Publication (Kokoku) No. 4-36735), and the mechanochemical method (Japanese Examined Patent Publication (Kokoku) No. 4-28412).

Generally, multilayer liposomes can be prepared by dissolving the below-mentioned phospholipids in a volatile organic solvent such as chloroform, methanol, dichloromethane, ethanol and the like, or a mixed solvent of said organic solvent and water, removing said solvent, and shaking or stirring the mixture.

In the step for removing solvent in the above-mentioned process, Bangham's method uses evaporation, but spray-drying or lyophilization also can be used.

In the above-mentioned liposome-preparing processes, the amount of the solvent used relative to lipid is not critical, and any amount which allows dissolution of lipid is acceptable. Removing solvent from the resulting mixture of lipid and solvent by evaporation can be carried out according to conventional procedure, such as evaporation under reduced pressure or, if necessary in the presence of inert gas. In practice, the above-mentioned volatile organic solvents may be used, if desired in mixed solvents comprising 10 volumes of said organic solvent and up to 1 volume of water.

To effect solvent removal by lyophilization, a solvent is selected which can be removed at a reduced pressure of about 0.005 to 0.1 Torr at a temperature lower than the freezing point of the solvent, typically −30° C. to −50° C. Where solvent removal is effected by spray drying the air pressure is typically controlled to 1.0 kg/cm² and the air flow rate to 0.35 cm²/minute, the inlet temperature being adjusted to a temperature higher than the boiling point of the solvent used. For example for the solvent chloroform, the temperature may be adjusted to 60 to 90° C., and the spray drying may be effected according to conventional procedures.

Several methods for the preparation of liposomes are known in the art and said methods may also be used for the preparation of the liposomes according to the invention (see for example D. D. Lasic et al., Preparation of liposomes. In D.D. Lasic (ed), Liposomes from physics to applications, Amsterdam, Elsevier Science Publishers B.V., The Netherlands, 1993, page 63- 107.) Methods known to the skilled artisan include for example the thin film hydration method and the reverse phase evaporation method. The liposomes according to the invention may be prepared by the thin film hydration method. Briefly, a chloroform/methanol solution of the phospholipids is rotary evaporated to dryness and the resulting film is further dried under vacuum (see for example D.D. Lasic, Preparation of liposomes. In D.D. Lasic (ed), Liposomes from physics to applications, Amsterdam, Elsevier Science Publishers B.V., The Netherlands, 1993, p. 67-73).

In a third step (c) the R¹ functional groups of the modified phospholipids of the liposomes are reacted with a group R²-Y. R¹ groups of modified phospholipids in the liposome membrane are exposed on the liposome surface and these R¹ groups are reacted with counter functional groups R² of R²-Y where R¹, R² and Y are described above. R^{1b} is a functional group which, under mild conditions such as aqueous buffer, reacts site-specific with R^{2b} yielding a stable conjugate. Respectively R^{2a} is a functional group which reacts site-specifically with R^{1a}. In this step a functional group of R^{1a} is reacted with a functional group R^{2a} or a functional group of R^{1b} is reacted with a functional group R^{2b} to give i) and ii) respectively

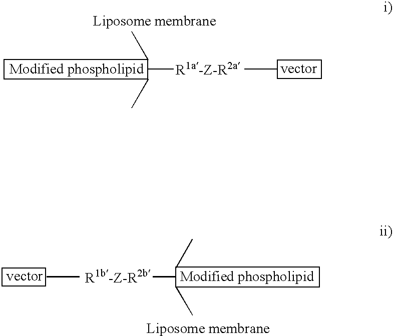

where Z is —CO—NH—, —NH—, —O—, —NHCONH—, or —NHCSNH—, and is preferably —CO—NH—, —NH—or —O—; R^{1a′}, R^{1b′}, R^{2a′} and R^{2b′} are the residues of R^{1a}, R^{1b}, R^{2a} and R^{2b} respectively after the conjugation reaction where Z is formed.

Suitably, an R^{1a} aldehyde in an amine containing phospholipid may be generated by oxidation of a precursor. Similarly, the R^{2b} aldehyde is generated by in situ oxidation of a precursor functionalised vector containing a 1,2-diol or 1,2 aminoalcohol group. For example, the latter can be inserted into a peptide sequence directly during synthesis using the amino acid Fmoc-Dpr(Boc-Ser)—OH described by Wahl et al in Tetrahedron Letts. 37, 6861 (1996).

Suitable oxidising agents which may be used to generate the R^{1a} or R^{2b} moiety in the amine containing phospholipid and R²-Y compound respectively, include periodate, periodic acid, paraperiodic acid, sodium metaperiodate, and potassium metaperiodate

R^{1a} and R^{2b} in the compounds above and related aspects of the invention are each preferably selected from —CHO, >C═O, —CH(—O—C₁₋₄alkyl-O—) such as —CH(—OCH₂CH₂O—), and —CH(OC₁₋₄alkyl)₂ such as —CH(OCH₃)₂, and in a preferred aspect R^{1a} and R^{2b} are —CHO.

R^{1b} and R^{2a} in the above compounds and related aspects of the invention are each preferably selected from —NHNH₂, —C(O)NHNH₂, and —ONH₂ and are preferably —ONH₂.

The reaction may be effected in a suitable solvent, for example, in an aqueous buffer in the pH range 2 to 11, suitably 3 to 11, more suitably 3 to 6, and at a non-extreme temperature of from 5 to 70° C., preferably at ambient temperature.

Phospholipids and mixtures thereof are the essential components for forming the membrane of liposomes. Examples of phospholipids and mixtures thereof that may be useful in the preparation of the liposomes of the present invention are neutral glycerophospho-lipids, for example a partially or fully hydrogenated naturally occurring (e.g. soybean- or egg yolk-derived) or synthetic phosphatidylcholine, particularly semi-synthetic or synthetic dipalmitoyl phosphatidylcholine (DPPC) or distearoyl phosphatidylcholine (DSPC), charged phospholipids include, for example, positively or negatively charged glycerophospholipids, negatively charged phospholipids include, for example, phosphatidylserine, for example a partially or fully hydrogenated naturally occurring (e.g. soybean- or egg yolk-derived) or semi-synthetic phosphatidylserine, particularly semi-synthetic or synthetic dipalmitoyl phosphatidylserine (DPPS) or distearoyl phosphatidylserine (DSPS); phosphatidylglycerol, for example a partially or fully hydrogenated naturally occurring (e.g. soybean- or egg yolk-derived) or semi-synthetic phosphatidylglycerol, particularly semi-synthetic or synthetic dipalmitoyl phosphatidylglycerol (DPPG) or distearoyl phosphatidylglycerol (DSPG); phosphatidylinositol, for example a partially or fully hydrogenated naturally occurring (e.g. soybean- or egg yolk-derived) or semi-synthetic phosphatidylinositol, particularly semi-synthetic or synthetic dipalmitoyl phosphatidylinositol (DPPI) or distearoyl phosphatidylinositol (DSPI); phosphatidic acid, for example a partially or fully hydrogenated naturally occurring (e.g. soybean- or egg yolk-derived) or semi-synthetic phosphatidic acid, particularly semi-synthetic or synthetic dipalmitoyl phosphatidic acid (DPPA) or distearoyl phosphatidic acid (OSPA), positively charged lipids include, for example, an ester of phosphatidic acid with an aminoalcohol, such as an ester of dipalmitoyl phosphatidic acid or distearoyl phosphatidic acid with hydroxyethylenediamine.

The liposomes of the present invention additionally comprise at least one amine containing phospholipid. Particularly preferred are phosphoethanolamines. Examples of preferred phosphoethanolamines are dipalmitoyl-glycero-3-phosphatidyethanolamine, myristoyl- palmitoyl-glycero-3-phosphoethanolamine, dimyristoyl-glycero-3-phosphoethanolamine, dipentadecanoyl-glycero-3-phosphoethanolamine, dipalmitoyl-glycero-3-phospho-ethanolamine, diheptadecanoyl-glycero-3-phospho-ethanolamine, distearoyl-glycero-3-phospho-ethanolamine, dinonadecanoyl-glycero-3-phosphoethanolamine and diarachidoyl-glycero-3-phosphoethanolamine, myristoyl-myristoleoyl-glycero-3-phospho-ethanolamine, myristoyl-myristelaidoyl-glycero-3-phosphoethanolamine, myristoyl- palmitoleoyl-glycero-3-phosphoethanolamine, myristoyl-palmitelaidoyl-glycero-3-phosphoethanolamine, myristoyl-oleoyl-glycero-3-phosphoethanolamine, myristoyl- elaidoyl-glycero-3-phosphoethanolamine, palmitoyl-myristoleoyl-glycero-3-phosphoethanolamine, palmitoyl-myristelaidoyl-glycero-3-phosphoethanolamine, palmitoyl-palmitoleoyl-glycero-3-phosphoethanolamine, palmitoyl-palmitelaidoyl-glycero-3-phosphoethanolamine, palmitoyl-oleoyl-glycero-3-phosphoethanolamine, palmitoyl-elaidoyl-glycero-3-phosphoethanolamine, palmitoyl-eicosenoyl-glycero-3-phosphoethanolamine, stearoyl-myristoleoyl-glycero-3-phosphoethanolamine, stearoyl-myristelaidoyl-glycero-3-phosphoethanolamine, stearoyl-palmitoleoyl-glycero-3-phosphoethanolamine, stearoyl-palmitelaidoyl-glycero-3-phospho-ethanolamine, stearoyl-oleoyl-glycero-3-phosphoethanolamine, stearoyl-elaidoyl-glycero-3-phosphoethanolamine, stearoyl-eicosenoyl-glycero-3-phosphoethanolamine, arachidoyl-palmitoleoyl-glycero-3-phosphoethanolamine, arachidoyl-palmitelaidoyl-glycero-3-phosphoethanolamine, arachidoyl-oleoyl-glycero-3-phosphoethanolamine, arachidoyl-elaidoyl-glycero-3-phospho-ethanolamine and arachidoyl-eicosenoyl -glycero-3-phosphoethanolamine.

Preferably, the liposomes comprise less than 10% of modified phospholipids. More preferably, less than 5% and most preferred less than 1% of modified phospholipids.

The liposomes may contain various optional components in addition to the above-mentioned components. For example, vitamin E (-tocopherol) and/or vitamin E acetate ester as an antioxidant may be added in an amount of 0.01 to 2 molar %, preferably 0.1 to 1 molar % relative to total amount of lipids.

By the term “vector” is meant any compound having binding affinity for a specific target e.g. receptor, tissue or cell type. Preferred biological vector of the present invention are peptides having binding affinity for a specific target e.g. receptor, tissue or cell type. In a preferred embodiment of the present invention the vector is a peptide comprising the Arg-Gly-Asp amino acid sequence or an analogue thereof such as those described in WO 01/77145 and WO 03/006491, preferably a peptide comprising the fragment

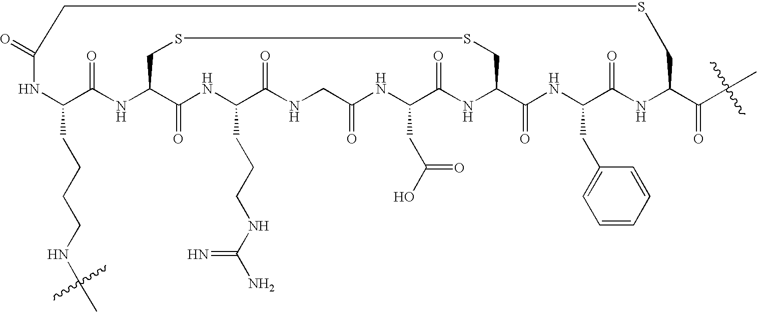

more preferably the peptide of formula (A):

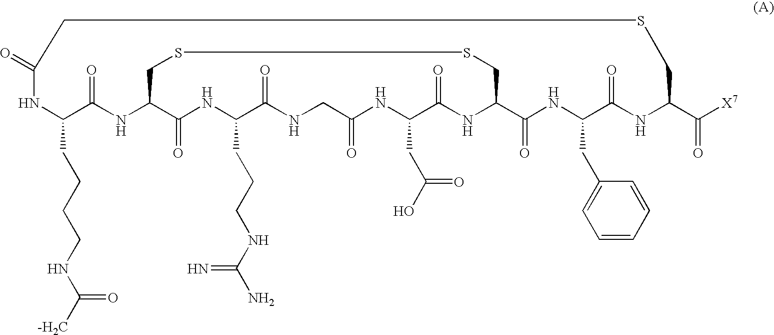

wherein X⁷ is either —NH₂ or

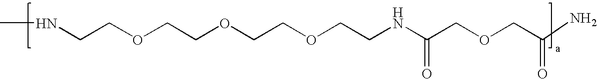

wherein a is an integer of from 1 to 10, preferably a is 1.

Optionally a Linker may be introduced between the amine containing phospholipid and the functional group R¹ and/or between R² and Y; (R²-Linker-Y).

It is envisaged that the role of the linker group is to distance the functional group on the amine containing phospholipids from the surface of the liposome to make the functional groups better available for reaction with the counter functional groups R². Further the role of linker group in the R²-Linker-Y moiety is to distance the vector (Y) from the relatively bulky liposome so that e.g. receptor binding is not impaired.

The Linker group is selected from

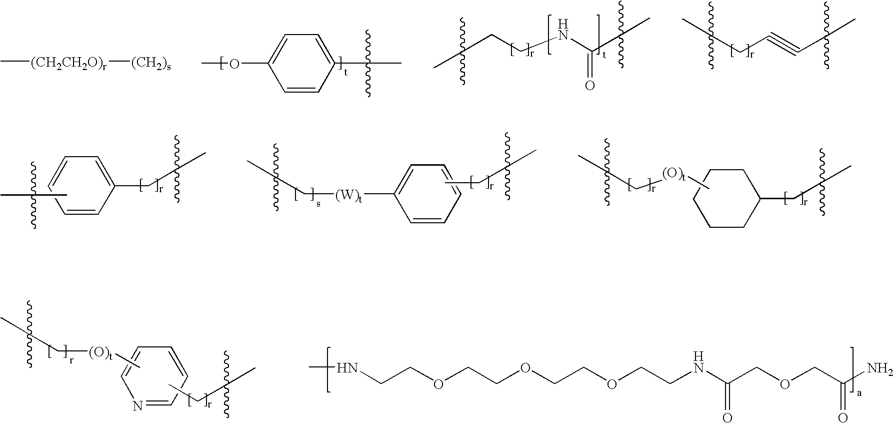

wherein:

r is an integer of 0 to 20;

s is an integer of 1 to 10;

t is an integer of 0 or 1;

a is an integer of from 1-10, preferably a is 1;

W is O or S.

The Linker groups are chosen to provide good in vivo pharmacokinetics, such as favourable excretion characteristics in the resultant conjugate. The use of linker groups with different lipophilicities and or charge can significantly change the in vivo pharmacokinetics of the liposome to suit the diagnostic need. For example, where it is desirable for a conjugate to be cleared from the body by renal excretion, a hydrophilic linker is used, and where it is desirable for clearance to be by hepatobiliary excretion a hydrophobic linker is used. Linkers including a polyethylene glycol moiety have been found to slow blood clearance which is desirable in some circumstances.

In an optional aspect of the present invention the liposomes can be manufactured so that the liposomes formed have in vivo imageable moieties bound to the membrane thereof preferably the imageable moieties are chelated diagnostically effective metal ions. Such liposomes can be manufactured as described in WO 96/11023 with the addition of modified phospholipids from step (a) of the present invention to the mixture of liposomal forming material; e.g. i) transforming a composition comprising an aqueous carrier medium, a liposomal membrane forming mixture which comprise modified phospholipids from step (a) of the present invention and a chelating agent having a hydrophobic membrane associating group attached thereto into a liposomal composition or ii) coupling a chelating agent to an anchor compound having a hydrophobic moiety incorporated within a liposomal membrane of a liposome comprising modified phospholipids from step (a) of the present invention. The chelating agents may then be metallated in a following step.

The modified phospholipids from step (a) of the present invention are added to the mixture of liposomal forming material in an amount of less than 10%, more preferred in an amount of less than 5%, most preferred in an amount of less than 1%.

The in vivo imageable moiety may be a chelate where the chelated compound for MRI is a paramagnetic metal, for SPECT, PET and scintigraphy an appropriate metal ion radioemitter and for X-ray a non-radioactive heavy metal ion.

Preferred radioisotopes for use in SPECT and scintigraphy are ⁹⁰Y, ^{99m}Tc, ¹¹¹In, ¹¹⁴In, ⁴⁷Sc, ⁶⁷Ga, ⁶⁸Ga, ⁸²Rb, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ²⁰¹Tl, ²⁰³Pb and ¹⁴¹ Ce. The choice of metal ion will be determined based on the desired diagnostic application.

For use in X-ray metal ions with atomic number greater than 37 and, in particular, metal ions with atomic number greater than 50 will be chelated. The in vivo imageable moiety preferably is a chelate where the chelated compound is a paramagnetic metal ion suitable for use in MRI.

The chelated compounds can be selected from ions of the transition and lanthanide metals having atomic numbers of 21-29, 42, 43, 44, or 57-71, preferred are ion of Cr, V, Mn, Fe, Co, Ni, Cu, La, Ce, Pr, Nd, Pm, Sm, Eu, Gd, Tb, Dy, Ho, Er, Tm, Yb and Lu, and particularly Gd-ions.

In a second aspect the invention provides a modified phospholipid where in the hydrophilic part of the phospholipid contains a group R¹ where R¹ is a functional group R^{1a} or R^{1b} where

R^{1a} is selected from an aldehyde moiety, a ketone moiety, a protected aldehyde as an acetal, a protected ketone such as a ketal, or a functionality such as diol or N-terminal serine residue, which can be oxidised to an aldehyde or ketone using an oxidising agent and

R^{1b} is a functional group which reacts site-specifically with R^{2b}-R^{1b} can be ammonia derivatives such as primary amine, secondary amine, hydroxylamine, hydrazine, hydrazide, aminoxy, phenylhydrazine, semicarbazide, or thiosemicarbazide, and is preferably a hydrazine, hydrazide, aminoxy, phenylhydrazine, semicarbazide or thiosemicarbazide group.

The modified phospholipids are preferably selected from the phosphoethanolamines described above.

The modified phospholipids can optionally comprise a linker as described above. Preferably the modified phospholipid is the modified phosphoethanolamine below.

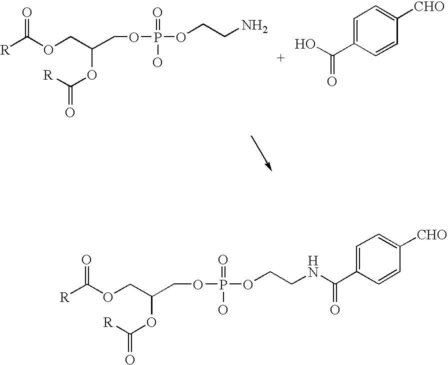

In a further aspect of the invention a modified phospholipid containing a vector is provided. Such phospholipids are the products of the conjugation of a phospholipid modified to contain a functional group R¹ with a R²-Y compound. The conjugation can be performed similarly to the conjugation described for step (c) above.

A further aspect of the invention provides liposomes containing modified phospholipids with R¹ functional groups attached thereto and further the inventions provides liposomes conjugated with R²-Y.

In a preferred embodiment the liposome of the invention is illustrated below:

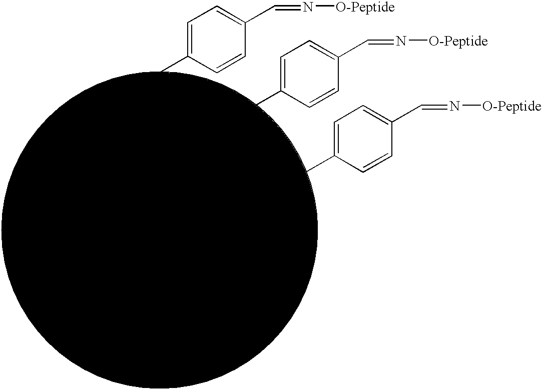

and where the peptide is (A)

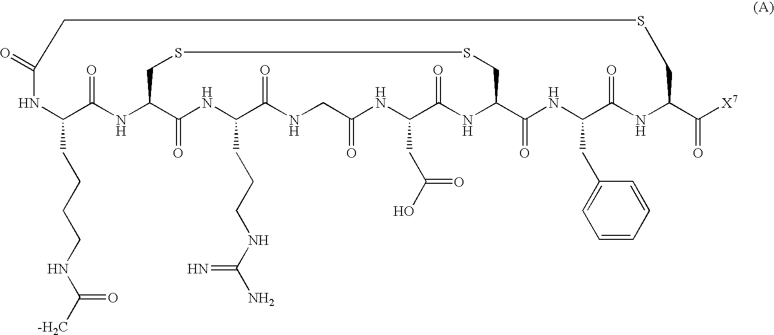

wherein X⁷ is either —NH₂ or

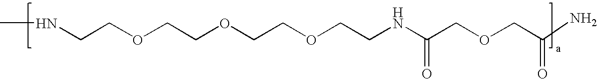

wherein a is an integer of from 1 to 10, preferably a is 1.

The present invention also provides a pharmaceutical composition comprising the liposome prepared by the process of the invention together with one or more pharmaceutically acceptable adjuvants, excipients or diluents.

Liposomes prepared by the process of the present invention are also valuable for medical use.

Further the liposome prepared by the process of the invention can be used for the manufacture of a MR contrast agent for the use in a method of in vivo imaging.

Liposome prepared by the process of the present invention are useful in methods of generating images of a human or animal body where said liposomes are administered to said body and images of at least a part of said body to which said liposome has distributed are generated using MRI.

The invention is illustrated by the non-limiting Examples detailed below.

The abbreviations used have the following meanings:

HATU —N—[(dimethylamino)-1H-1,2,3-triazolo[4,5-b]pyridino-1-ylmethylene]-N-methylmethanaminium hexafluorophosphonate N-oxide

Boc—t-butyloxycarbonyl

DMF—dimethylformamide

DSPE—distearoylphosphatidylethanolamine

DPPC—dipalmitoyl phosphatidylcholine

DPPG—dipalmitoyl phosphatidylglycerol

PyAOP—7-Azabenzotriazol-1-yloxytris(pyrrolidino)phosphonium-hexafluorophosphate

NMM—N-methylmorpholine

TFA—trifluoractic acid

PEG—Poly ethylene glycol

SC—succinimidyl carbonate

### EXAMPLES

### 1a) Synthesis of Boc-NH-PEG₃₄₀₀-DSPE(t-Butyl Carbamate Poly(Ethylene Glycol)Distearoylphosphatidyl-Ethanolamine)

DSPE (distearoylphosphatidylethanolamine) (31 mg, Sygena Inc.) was added to a solution of Boc-NH-PEG₃₄₀₀-SC (t-butyl carbamate poly(ethylene glycol)-succinimidyl carbonate) (150 mg) in chloroform (2 ml), followed by triethylamine (33 μl). The mixture formed a clear solution after stirring at 41° C. for 10 minutes. The solvent was rotary evaporated and the residue taken up in acetonitrile (5 ml). The thus-obtained dispersion was cooled to 4° C. and centrifuged, whereafter the solution was separated from the undissolved material and evaporated to dryness. The structure of the resulting product was confirmed by NMR.

### 1b) Synthesis of H₂N-PEG₃₄₀₀-DSPE(Amino-Poly(Ethylene Glycol)-Distearoylphosphatidylethanolamine)

Boc-NH-PEG₃₄₀₀-DSPE (167 mg) was stirred in 4 M hydrochloric acid in dioxane (5 ml) for 2.5 hours at ambient temperature. The solvent was removed by rotary evaporation and the residue was taken up in chloroform (1.5 ml) and washed with water (2×1.5 ml). The organic phase was removed by rotary evaporation. TLC (chloroform/methanol/water 13:5:0.8) gave the title product with Rf=0.6; the structure of the product, which was ninhydrin positive, was confirmed by NMR.

### 1c) Synthesis of 4-Formylbenzenamido-PEG₃₄₀₀-DSPE

To a solution of 4-carboxybenzaldehyde (2.7 mg, 0.018 mmol) and HATU (6.8 mg, 0.018 mmol) in DMF (1 ml) is added diethylisopropylamine (6.2 μl, 0.036 mmol). The mixture is stirred at room temperature for 5 min and added to a solution of H₂N-PEG₃₄₀₀-DSPE from b)(65 mg, 0.012 mmol) in DMF (1 ml). The reaction mixture is stirred for 12 hrs and concentrated (rotavapor). The residue is purified by flash chromatography (silica, chloroform/methanol/water). The product is isolated by evaporation of the solvents.

### 1d) Preparation of DPPC/DPPG/4-Formylbenzenamido-PEG₃₄₀₀-DSPE Liposomes

DPPC/DPPG/4-Formylbenzenamido-PEG₃₄₀₀-DSPE liposomes, with a weight ratio of lipids at 90/5/5 are prepared by the thin film hydration method. The lipids (500mg) are mixed in chloroform and methanol and evaporated to dryness at reduced pressure. The lipid film is shaken in a saline solution (10 ml) at 57° C. form vesicles and the liposomes are in additional subjected to 3 freeze-thaw cycles. The resultant large vesicles are then extruded under pressure through polycarbonate filters of various pore diameters using an extruder preheated at 65° C. The resultant liposomes after extrusion have a particle diameter of 70 nm. The saline solution is exchanged with 0.1 M NH₄OAc buffer, pH4, by dialysis.

### 1e) Conjuqation of Aminooxy Modified Peptide (Compound 15 in Structure Formula Below) to Liposomes Comprising 4-Formylbenzenamido-PEG₃₄₀₀-DSPE

The peptide, Compound 14 in structure formula below was synthesised using standard peptide synthesis. Compound 14 in structure formula below (150 mg, 0.12 mmol) in DMF was added to a solution of Boc-aminoxyacetic acid (34.4 mg, 0.18 mmol), PyAOP (93.9 mg, 0.18mmol) and NMM (40 μl, 0.36 mmol) in DMF. DMF was evaporated under reduced pressure after 12 hours and the crude product was purified by reverse phase preparative chromatography (Phenomenex Luna C18 column, OOG-4253-V0; solvents A=water/0.1% TFA and B=CH₃CN/0.1% TFA; gradient 10-50% B over 60 min; flow 50 ml/minute; detection at 254 nm), affording 97.1 mg (57%) of pure compound (analytical HPLC: Phenomenex Luna C18 column, 00G-4252-E0; solvents: A=water+0.1% TFA/B=CH₃CN+0.1% TFA, gradient: 10-50% B over 20 min; flow 1.0 ml /minute; retention time 19.4 minutes, detected at 214 and 254 nm). Further characterisation was carried out using mass spectrometry, giving m/z value 1431.2 [M-H+].

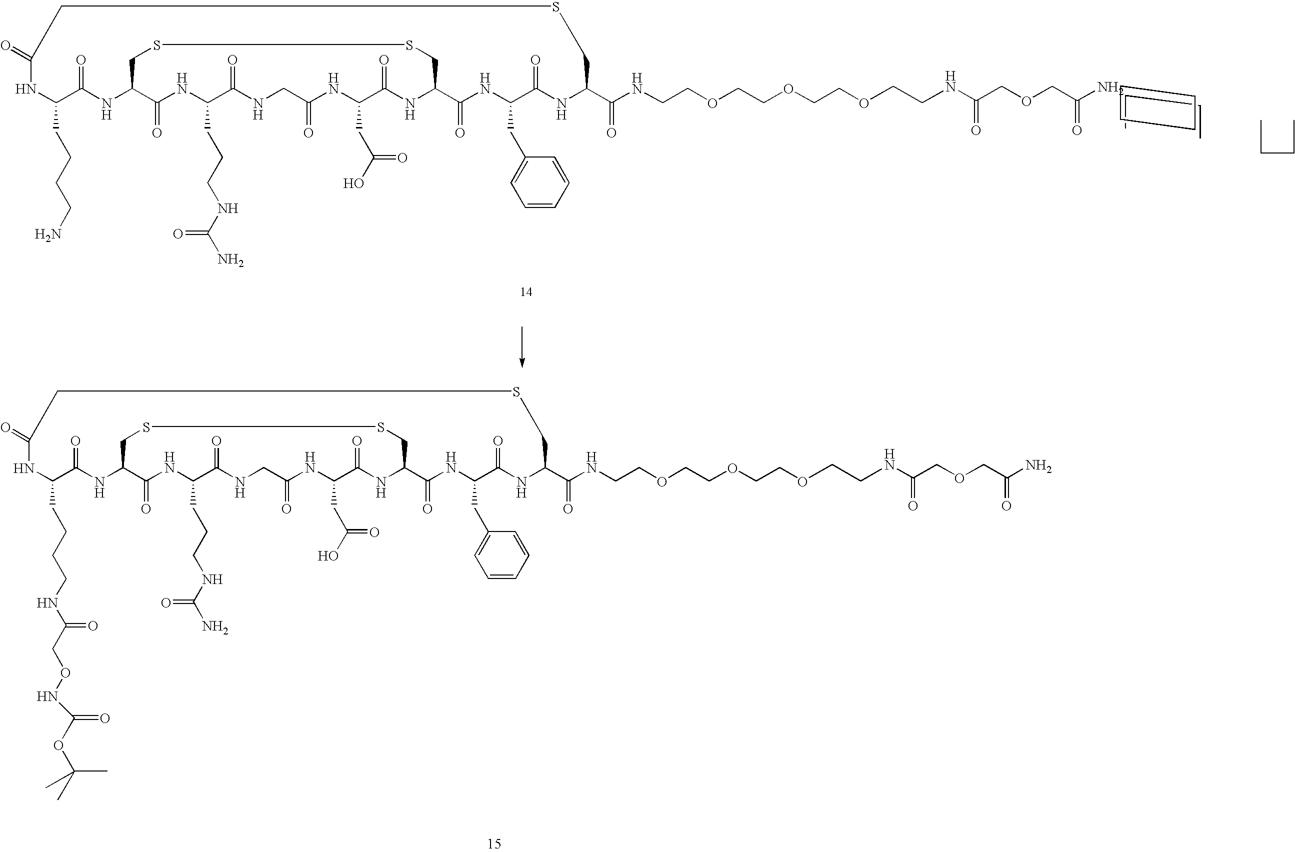

Boc protected peptide (compound 15 in structure formula above) (12 mg) is treated with 5% water in TFA (1 ml) for 5 min at room temperature. The solvents are removed by evaporation under vacuum. The deprotected peptide is redissolved in 0.1 M NH₄OAc buffer, pH4 (0.5 ml), combined with liposome suspension from d) and heated at 70° C. for 15 min. After cooling to room temperature the liposome suspension is dialysed against an isoosmotic PBS solution.

1. A process for the manufacture of targeting liposomes comprising vector compounds conjugated to the hydrophilic part of modified phospholipids characterised in
(a) reacting an amine containing phospholipid with a group R¹-X wherein R¹ is a functional group R^{1a} selected from an aldehyde moiety, a ketone moiety, a protected aldehyde as an acetal, a protected ketone such as a ketal, or a functionality such as diol or N-terminal serine residue, which can be oxidised to an aldehyde or ketone using an oxidising agent and X is a reactive group that in the reaction with the amine of the phospholipid forms an amide bond by which a modified phospholipid containing a functional group R¹ is formed,
(b) forming liposomes optionally comprising in vivo imageable moieties bound to the membrane from a mixture comprising the modified phospholipids from (a) in a conventional manner, and
(c) reacting the R¹ functional groups of the modified phospholipids of the liposomes with a group R²-Y wherein R² is a functional group R^{2a} selected from primary amine, secondary amine, hydroxylamine, hydrazine, hydrazide, aminoxy, phenylhydrazine, semicarbazide or thiosemicarbazide group and
Y is a vector,
to form targeting liposomes.

2. A process according to claim 1 characterised in that X is an acidic group, an anhydride or an ester.

3. A process according to claim 1 characterised in that
in step (a) the functional group R¹ is an aldehyde containing moiety and X is a group —COOH and in step (c) the functional group R² is an aminoxy containing moiety.

4. A process according to claim 1 characterised in that the amine containing phospholipid is a phosphoethanolamine.

5. A process according to claim 1 characterised in that said amine containing phospholipids are present in the liposome in an amount of less than 10%.

6. A process according to claim 1 characterised in that said amine containing phospholipids are present in the liposome in an amount of less than 5%.

7. A process according to claim 1 characterised in that said amine containing phospholipids are present in the liposome in an amount of less than 1%.

8. A process according to claim 1 characterised in that the process further comprises the step where a liposome containing an in vivo imageable moiety is obtained.

9. A process according to claim 8 characterised in that said in vivo imageable moiety is a chelate wherein the chelated compound is a paramagnetic metal ion suitable for use in MRI.

10. A process according to claim 9 characterised in that said chelated compound is an ion of the transition and lanthanide metals having atomic numbers of 21-29, 42, 43, 44, or 57-71.

11. A process according to claim 9 characterised in that said chelated compound is an ion of Cr, V, Mn, Fe, Co, Ni, Cu, La, Ce, Pr, Nd, Pm, Sm, Eu, Gd, Tb, Dy, Ho, Er, Tm, Yb and Lu, and particularly Gd-ions.

12. A phospholipid characterised in that the hydrophilic part of said phospholipid contains a functional group R¹ wherein R¹ is R^{1a} selected from an aldehyde moiety, a ketone moiety a protected aldehyde as an acetal, a protected detone such as a ketal, or a functionality such as diol or N-terminal serine residue, which can be oxidised to an aldehyde or ketone using an oxidizing agent.

13. A phospholipid according to claim 12 characterised in that said phospholipid is a modified phosphoethanolamine.

14. A phospholipid according to claim 12 characterised in that the functional group R¹ is distanced from the hydrophilic part of said phospholipid by a linker.

15. (canceled)

16. A liposome characterised in that the membrane of said liposome contains phospholipids of claim 12.

17. A liposome characterised in that a vector (Y) is covalently bound to the phospholipids of claim 12 of liposome surface.

18. A liposome according to claim 17 characterised in that a functional group R^{1a} at the liposome surface is conjugated with the functional group R^{2a} of the group R^{2a}—Y to form the conjugate R^{1a′}p—Z—R^{2a′}—Y where R^{1a} is selected from primary amine, secondary amine, hydroxylamine, hydrazine, hydrazide, aminoxy, phenylhydrazine, semicarbazide or thiosemicarbazide group,
Y is a vector,
Z is —CO—NH—, —NH—, —O—, —NHCONH—, or —NHCSNH—, and
R^{1a′} and R^{2a′} are the residues of R^{1a} and R²a respectively after the conjugation reaction where Z is formed.

19. (canceled)

20. A liposome according to claim 17 characterised in that the functional group R¹ is benzaldehyde, R² is an aminoxy and Y is peptide comprising the fragment

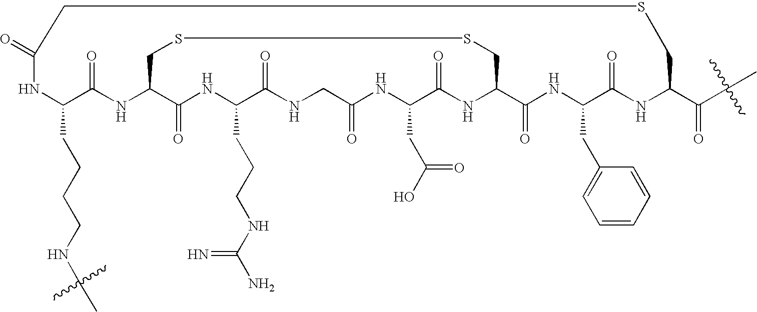

21. A liposome according to claim 17 characterised in that Y is a peptide of formula (A) 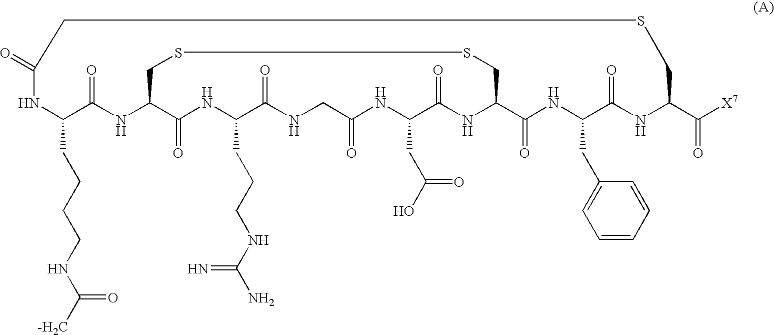 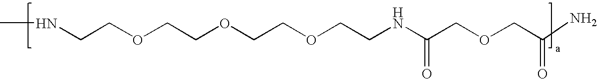
wherein X⁷ is either —NH₂ or
wherein a is an integer of from 1 to 10, preferably a is 1.

22. A liposome according to claim 17 characterised in that the liposome comprise an in vivo imageable moiety.

23. A liposome according to claim 22 characterised in that said in vivo imageable moiety is a chelate wherein the chelated compound is a paramagnetic metal ion suitable for use in MRI.

24. A liposome according to claim 22 characterised in that said chelated compound is an ion of the transition and lanthanide metals having atomic numbers of 21-29, 42, 43, 44 or 57-71.

25. A liposome according to claim 22 characterised in that said chelated compound is an ion of Cr, V, Mn, Fe, Co, Ni, Cu, La, Ce, Pr, Nd, Pm, Sm, Eu, Gd, Tb, Dy, Ho, Er, Tm, Yb and Lu, and particularly Gd-ions.

26. A pharmaceutical composition comprising the liposome as claimed in claim 17 together with one or more pharmaceutically acceptable adjuvants, excipients or diluents.

27. A liposome as claimed in claim 17 for medical use.

28. Use of a liposome as claimed in claim 17 for the manufacture of a MR contrast agent for the use in a method of in vivo imaging.

29. A method of generating an image of a human or animal body comprising administering a liposome as claimed in claim 22 to said body and generating an image of at least a part of said body to which said liposome has distributed using MRI.
